(19) United States
(12) Patent Application Publication
(10) Pub. No.: US 2026/0038655 A1
(43) Pub. Date: Feb. 5, 2026

(54) SYSTEMS AND METHODS FOR AUTOMATED HEALTH CARE JOURNEY MANAGEMENT

(71) Applicant: Medecision, Inc., Dallas, TX (US)

(72) Inventor: Kenneth Young, Blue Bell, PA (US)

(21) Appl. No.: 19/285,648

(22) Filed: Jul. 30, 2025

Related U.S. Application Data
(60) Provisional application No. 63/677,797, filed on Jul. 31, 2024.

(51) Int. Cl. G16H 10/60; G16H 15/00; G16H 20/00; G16H 50/30; G16H 50/70; G16H 80/00

(52) U.S. Cl. CPC G16H 10/60; G16H 15/00; G16H 20/00; G16H 50/30; G16H 50/70; G16H 80/00

(57) ABSTRACT

Methods, non-transitory computer readable media, and care management systems are disclosed that filter member data based on population parameter data to identify members for a program corresponding to a disease state or medical condition. A journey is generated for the program based on orchestration data defining an automated engagement with the members and obtained from a care manager device via provided GUIs. The journey is executed based on the orchestration data. A determination is made that an action associated with one of the members has occurred. The journey is modified for the member by applying rules to action data to generate other orchestration data defining another automated engagement with the member. The modified journey is executed based on the second orchestration data. Executing the journey or the modified journey includes sending via communication networks check-in messages, assessments, or educational content at selected times to member devices associated with the members.

This application claims priority to U.S. Provisional Patent Application Ser. No. 63/677,797, filed Jul. 31, 2024, which is hereby incorporated by reference herein in its entirety.

### TECHNICAL FIELD

This technology generally relates to patient care coordination and management systems and, more particularly, to digital health care journey management, communication, and automation systems and methods that assist care managers in orchestrating patient engagement, collaboration, and support services.

### BACKGROUND

Efficient and effective care coordination and patient engagement remains elusive for health plans and health care managers due to manual communication prompting, poor quality data, and/or inadequate workflow technologies, for example. Legacy care management solutions are widely recognized to be deficient because they were not designed to succeed at the speed and complexity of the current healthcare marketplace. Current care management systems are cumbersome to use, fail to implement automated actions (e.g., patient communications or educational content delivery) responsive to real-time data, and do not effectively orchestrate engagement, resulting in gaps in care, higher utilization rates, higher health care costs, and reduced health outcomes for patients.

### BRIEF DESCRIPTION OF THE DRAWINGS

FIG. 1 is a block diagram of an exemplary network environment with a care management system;

FIG. 2 is a block diagram of an exemplary care management system;

FIG. 3 is a flowchart of an exemplary method for automated health care journey management;

FIG. 4 is an exemplary program generation interface;

FIG. 5 is an exemplary program selection interface;

FIG. 6 is an exemplary population selection interface;

FIG. 7 is an exemplary opt-in orchestration interface;

FIG. 8 is an exemplary assessment delivery interface;

FIG. 9 is an exemplary assessment reminder interface;

FIG. 10 is an exemplary assessment completion interface;

FIG. 11 is an exemplary active journey check-in interface;

FIG. 12 is an exemplary active journey education content delivery interface;

FIG. 13 is an exemplary active journey pause interface;

FIG. 14 is an exemplary exit journey interface;

FIG. 15 is an exemplary review and launch interface;

FIG. 16 is an exemplary member program care manager dashboard;

FIG. 17 is an exemplary member assessment care manager dashboard;

FIG. 18 is an exemplary assessment member dashboard; and

FIG. 19 is an exemplary check-in member dashboard.

### DETAILED DESCRIPTION

This technology provides several advantages including methods, non-transitory computer readable media, and care management systems that advantageously orchestrate automated digital health care journeys for patients, which are also referred to herein as members. The disclosed technology provides a platform with improved, intuitive graphical user interfaces (GUIs) that allow care managers to establish and deliver personalized, optimized health experiences and proactively engage and influence members to manage their health effectively, while lowering total costs.

Care management systems in accordance with examples of this technology orchestrate omnichannel communications, provide adaptive journey modifications responsive to real-time data and actions, enable journey self-editing capabilities, and deliver and track consumption of educational content. By more effectively engaging members during health care journeys, this technology enables organizations to identify at-risk and rising-risk individuals more effectively and proactively and empowers them to achieve better outcomes through collaboration across the care continuum.

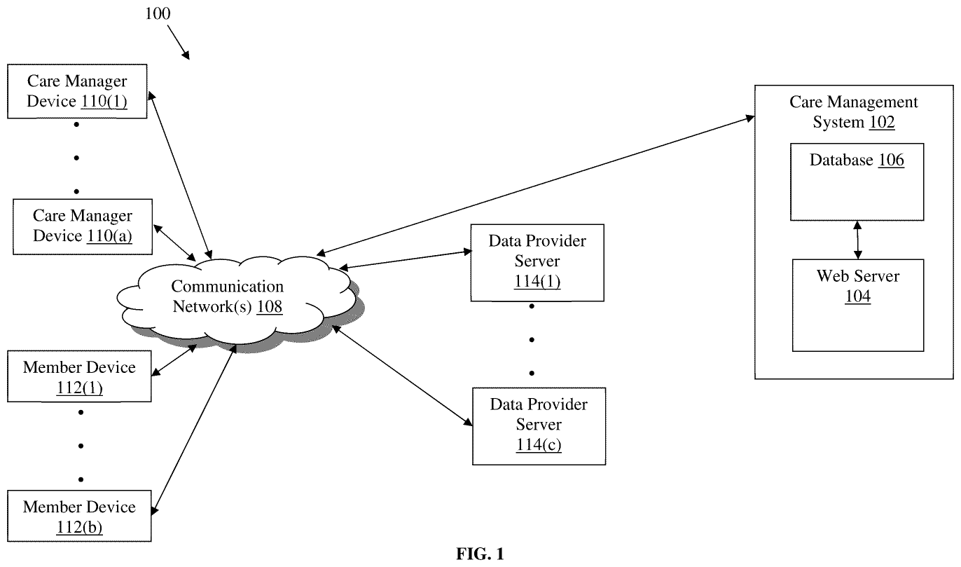

Referring to FIG. 1, an exemplary network environment is illustrated that includes a care management system 102, with a web server 104 coupled to a backend database 106, which is coupled via communication network(s) 108 to care manager devices 110(1)-110(a), member devices 112(1)-112(b), and data provider servers 114(1)-114(c). The network environment may include other network devices such as one or more routers or switches, for example, which are known in the art and thus will not be described herein.

In this example, the care manager devices 110(1)-110(a), member devices 112(1)-112(b), data provider servers 114(1)-114(c), web server 104, and database 106 are disclosed in FIG. 1 as dedicated hardware devices. However, one or more of the care manager devices 110(1)-110(a), member devices 112(1)-112(b), data provider servers 114(1)-114(c), web server 104, or database 106 can also be implemented in software within one or more other devices in the network environment 100. As one example, the database 106, as well as any of its components or applications, can be implemented as software executing on the web server 104, and many other permutations and types of implementations and network topologies can also be used in other examples.

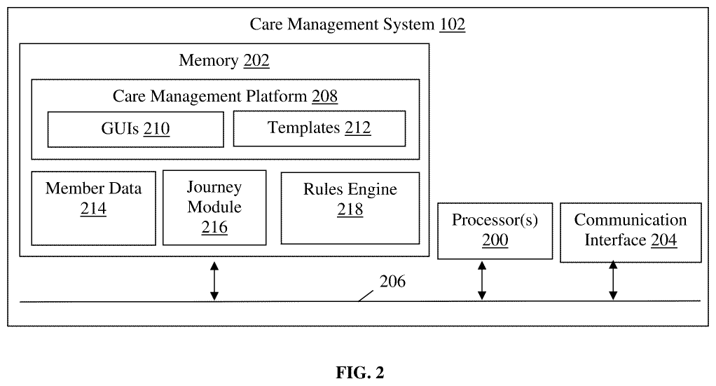

Referring to FIGS. 1-2, the care management system 102 may perform any number of functions, including providing GUIs to the care manager devices 110(1)-110(a), orchestrated delivery of communications and educational content to the member devices 112(1)-112(b), and other functions as described and illustrated in detail below. The care management system 102 (e.g., the web server 104 of the care management system 102) in this example includes processor(s) 200, memory 202, and a communication interface 204, which are coupled together by a bus 206, although the care management system 102 can include other types or numbers of elements in other configurations.

The processor(s) 200 of the care management system 102 may execute programmed instructions stored in the memory 202 of the care management system 102 for any number of the functions described and illustrated herein. The processor(s) 200 may include one or more general purpose processors with one or more processing cores, one or more central processing units, and/or one or more graphics processing units, for example, although other types of processor(s) can also be used.

The memory 202 stores these instructions for one or more aspects of the present technology as described and illustrated herein, although some or all of the programmed instructions could be stored elsewhere. A variety of different types of memory storage devices, such as random-access memory (RAM), read only memory (ROM), hard disk, solid state drives, flash memory, or other computer readable medium which is read from and written to by a magnetic, optical, or other reading and writing system that is coupled to the processor(s) 200, can be used for the memory 202.

Accordingly, the memory 202 can store applications that can include computer executable instructions that, when executed by the processor(s) 200, cause the care management system 102 to perform actions, such as to transmit, receive, or otherwise process network messages and requests, for example, and to perform other actions described and illustrated below. The application(s) can be implemented as components of other applications, operating system extensions, and/or plugins, for example.

Further, the application(s) may be operative in a cloud-based computing environment with access provided via a software-as-a-service (SaaS) model. The application(s) can be executed within or as virtual machine(s) or virtual server(s) that may be managed in a cloud-based computing environment. Also, the application(s), and even the care management system 102 itself, may be located in virtual server(s) running in a cloud-based computing environment rather than being tied to specific physical network computing devices. Also, the application(s) may be running in virtual machines (VMs) executing on the care management system 102 and managed or supervised by a hypervisor.

In this example, the memory 202 includes a care management platform 208 with GUIs 210 and templates 212, member data 214, a journey module 216, and a rules engine 218. In some examples, the member data 214 can be maintained in the database 106 of the care management system 102, which can be a relational database (e.g., a Structured Query Language (SQL) database), although other types of databases can also be used in other examples. The member data 214 can be obtained from the data provider servers 114(1)-114(c) (e.g., using provided application programming interfaces (APIs) or other endpoints) and can include demographic data, a generated unique identifier, an indication of social determinants of health (SDoH) needs or SDoH base data, one or more SDoH risk scores, clinical data, admission, discharge, and transfer (ADT) data, and/or an indication of an associated care manager (e.g., a unique identifier of the care manager), although other member data 214 can also be used in other examples.

The care management platform 208 is configured to generate and provide the GUIs 210 to the care manager devices 110(1)-110(a) to facilitate orchestration of member engagement, such as via electronic communications and delivery of assessments and educational content. The templates 212 can be enterprise-specific language useful for types of communications as part of the orchestration, and modifiable by users of the care manager devices 110(1)-110(a), as explained in more detail below. Thus, the care management platform 208 is configured to establish programs, populations within the programs, and journeys within the program, with the journeys being based on the orchestration of engagement, also as described and illustrated in more detail below.

The journey module 216 can be configured to automatically carry out journeys as generated by the users of the care manager devices 110(1)-110(a) via the care management platform 208. The journey module 216 can also monitor for real-time actions (e.g., assessment submission, ADT data associated with a member, or receipt of clinical data) and leverage the rules engine 218 to apply artificial intelligence rules to modify a journey established for an associated member. Thus, the rules engine 218 defines real-time responses to actions observed by the care management system 102. In some examples, actions can be identified based on data obtained from the data provider server 114(1)-114(c) and in other examples the actions can be based on an interaction received from one of the member devices 112(1)-11(b), for example.

Accordingly, the journey module 216 manages the journey for a member, as orchestrated by a care manager, as well as the dynamic modification of the journey. The management of the journey can include automated communications with the member devices 112(1)-112(b) via the communication network(s) 108 and/or educational content delivery via the care management platform 208, for example.

The communication interface 204 of the care management system 102 operatively couples and communicates between the care management system 102, care manager devices 110(1)-110(a), member devices 112(1)-112(b), and data provider servers 114(1)-114(c), which are coupled together at least in part by the communication network(s) 108 in this particular example, although other types or numbers of communication networks or systems with other types or numbers of connections or configurations to other devices or elements can also be used. The communication network(s) 108 can include wide area network(s) (WAN(s)) and/or local area network(s) (LAN(s)), for example, and can use TCP/IP over Ethernet and industry-standard protocols, although other types or numbers of protocols or communication networks can be used. The communication network(s) 108 can employ any suitable interface mechanisms and network communication technologies including, for example, Ethernet-based Packet Data Networks (PDNs).

The care management system 102 in some examples can include a plurality of devices each having one or more processors (each processor with one or more processing cores) that implement one or more steps of this technology. In these examples, one or more of the devices can have a dedicated communication interface or memory. Alternatively, one or more of the devices can utilize the memory 202, communication interface 204, or other hardware or software components of one or more other devices included in the care management system 102. Additionally, one or more of the devices that together comprise the care management system 102 in other examples can be standalone devices or integrated with one or more other devices or apparatuses.

Each of the data provider servers 114(1)-114(c) can include processor(s), memory, and a communication interface, which are coupled together by a bus or other communication link (not illustrated), although other numbers or types of components could also be used. The data provider servers 114(1)-114(c) can be associated with or hosted by SDoH service providers, clinical laboratories, ADT data providers, and/or insurance providers, for example. Accordingly, the data provider servers 114(1)-114(c) can provide API endpoints, for example, which allow the care management system 102 to obtain the member data useful for population identification as well as for real-time journey modification, as explained in more detail below.

Each of the care manager devices 110(1)-110(a) and member devices 112(1)-112(b) of the network environment 100 in this example includes any type of computing device that can exchange network data, such as mobile, desktop, laptop, or tablet computing devices, virtual machines (including cloud-based computers), or the like. Each of the care manager devices 110(1)-110(a) and member devices 112(1)-112(b) in this example includes processor(s), memory, and a communication interface, which are coupled together by a bus or other communication link (not illustrated), although other numbers or types of components could also be used.

Each of the care manager devices 110(1)-110(a) and member devices 112(1)-112(b) may run interface applications, such as web browsers or standalone applications, which may provide an interface to communicate with the care management system 102 via the communication network(s) 108. Each of the care manager devices 110(1)-110(a) and member devices 112(1)-112(b) may further include a display device, such as a display screen or touchscreen, or an input device, such as a keyboard or mouse, for example (not shown). One or more of the care manager devices 110(1)-110(a) and member devices 112(1)-112(b) may be associated with an individual user (e.g., a care manager or a member, respectively), for example. The care manager devices 110(1)-110(a) can be used by care managers to interface with the care management system 102 to manage care and orchestrate engagement for associated members.

Although the exemplary network environment 100 with the care manager devices 110(1)-110(a), member devices 112(1)-112(b), data provider servers 114(1)-114(c), care management system 102, and communication network(s) 108 are described and illustrated herein, other types or numbers of systems, devices, components, or elements in other topologies can be used. It is to be understood that the systems of the examples described herein are for exemplary purposes, as many variations of the specific hardware and software used to implement the examples are possible, as will be appreciated by those skilled in the relevant art(s).

One or more of the components depicted in the network environment, such as the care manager devices 110(1)-110(a), member devices 112(1)-112(b), data provider servers 114(1)-114(c), or care management system 102, for example, may be configured to operate as virtual instances on the same physical machine. In other words, one or more of the care manager devices 110(1)-110(a), member devices 112(1)-112(b), data provider servers 114(1)-114(c), or care management system 102 may operate on the same physical device rather than as separate devices communicating through the communication network(s) 108. Additionally, there may be more or fewer care manager devices, service provider devices, service provider servers, or care coordination systems than illustrated in FIG. 1.

The examples of this technology may also be embodied as one or more non-transitory computer readable media having instructions stored thereon, such as in the memory 202 of the care management system 102, for one or more aspects of the present technology, as described and illustrated by way of the examples herein. The instructions in some examples include executable code that, when executed by one or more processors, such as the processor(s) 200 of the care management system 102, cause the processors to carry out steps necessary to implement the methods of the examples of this technology that will now be described and illustrated herein.

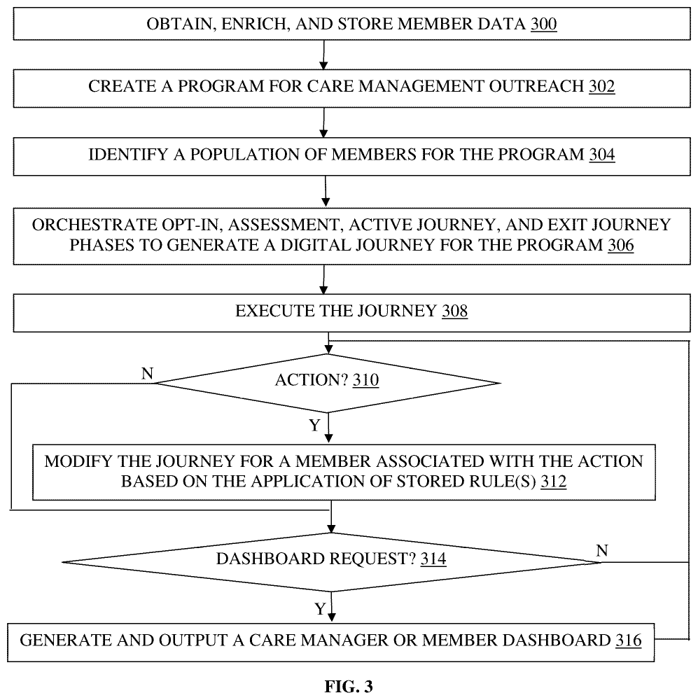

Referring now to FIG. 3, a flowchart of an exemplary method for automated health care journey management is illustrated. In step 300 in this example, the care management system 102 obtains, enriches, and stores the member data 214. The member data 214 in this example includes base member data and enriched member data that is generated based on an analysis of the base member data. The base member data can include one or more of an association of members with care managers, demographic data for members (e.g., patients), clinical data (e.g., lab, pharmacy, authorization, diagnosis, and/or procedure information), ADT data, member history data, SDoH need data, and/or any other information.

In some examples, the base member data is ingested from the care manager devices 110(1)-110(a) (e.g., using forms provided by the care management system 102 or via provided electronic files that are automatically parsed and processed) and, in other examples, the base member data can be ingested via an integration with an electronic medical record (EMR) system associated with a care manager, for example, which can be one of the data provider servers 114(1)-114(c). Other methods of obtaining the base member data can also be used.

The association of members with care managers can be based on unique identifiers for the members and the care managers. The unique identifiers for the members can be generated by the care management system 102 upon ingest and maintained in the member data 214. The care management system 102 can register the care managers based on login credentials, which are stored in the memory 202 and can include unique identifiers (e.g., usernames) for the care managers, or the unique identifiers for the care managers also can be generated by the care management system 102 following registration. Additionally, the association of members to care managers can be automatic based on the ingest process or manually established by care manager users of the care manager devices 110(1)-110(a), for example.

The care management system 102 also obtains or generates enriched member data, such as SDoH risk scores for each of the members, which is stored in the member data 214. The SDoH risk scores can include scores for categories such as transportation, food, pharmacy, or behavioral health, and/or an overall SDoH risk score, for example. The care management system 102 can ingest the SDoH risk scores from a third-party source or generate the SDoH risk scores from the base member data associated with the particular members, for example.

Accordingly, the SDoH risk scores can be indicative of a need for a particular member with respect to a particular SDoH category (e.g., transportation, food, pharmacy, or behavioral health). Exemplary methods for generating SDoH risk scores are disclosed in U.S. Patent Application Publication No. 2024/0347178, published Oct. 17, 2024, which is hereby incorporated by reference herein in its entirety. Other types of enriched member data can also be generated and/or stored with the member data 214 in other examples.

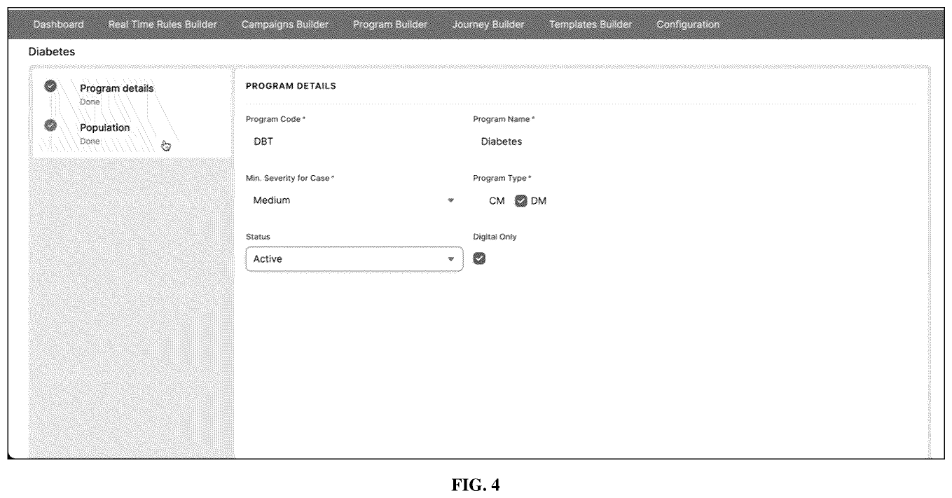

In step 302, the care management system 102 creates a program for care management outreach based on input received from one of the care manager device 110(1)-110(a) obtained via one of the GUIs 210. The programs in this example each correspond to one or more disease states or medical conditions and have a one-to-one relationship with journeys (e.g., digital multistep campaigns) that are described and illustrated in more detail below. Referring to FIG. 4 an example of the GUIs 210, which can be used to create a programs, is illustrated in the form of a program generation interface. In this example, a user of one of the care manager devices 110(1)-110(a) can interact with the program generation interface to input a program code, program name, minimum severity, type, status, and an indication of whether the program is digital only, although other program data associated with a created program can also be obtained in step 302.

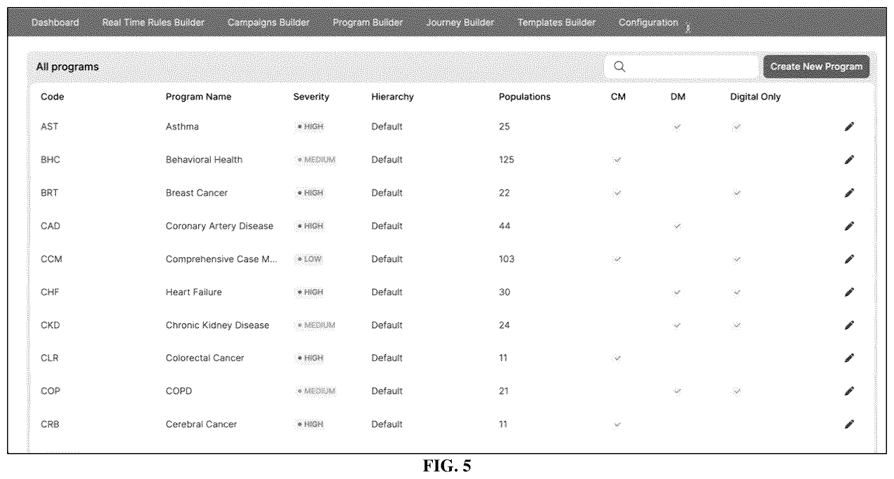

In some examples, the program data can be stored in the memory 202 to facilitate re-use via a program selection interface one of the GUIs 210, an example of which is illustrated in FIG. 5. In this example, customized programs having stored program data are selectable and searchable to allow a care manager to re-use the program data, adjust the program data for an active program, or view details regarding a program, for example.

Referring back to FIG. 3, in step 304, the care management system 102 identifies a population of members (also referred to as a cohort) for the program for care management outreach created in step 302. In some examples, the care management system 102 identifies the population based on input received from one of the care management devices 110(1)-110(a) to a provided one of the GUIs 210. The one of the GUIs 210 allows a care manager to input population parameter data subsequently used by the care management system 102 to automatically filter the stored member data 214 and thereby identify the population for the created program.

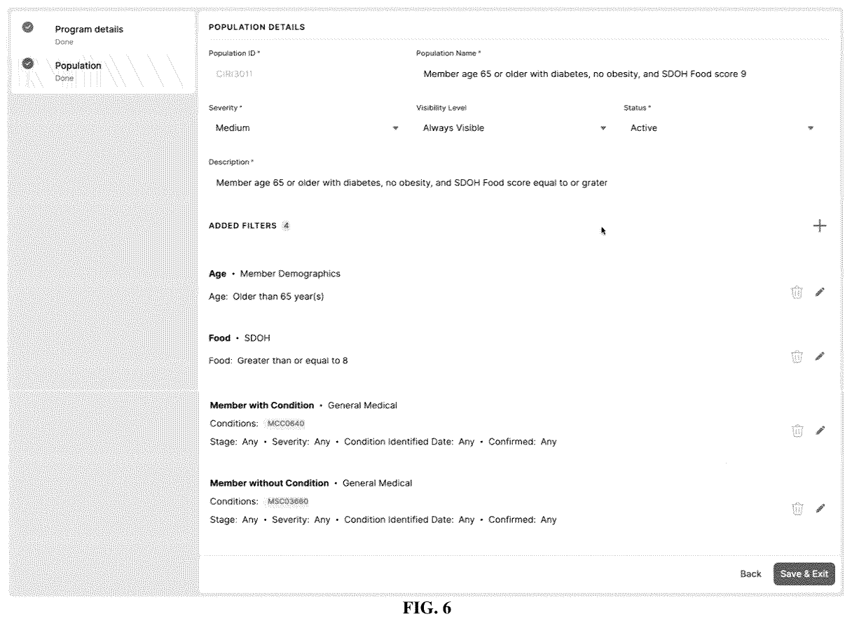

Referring to FIG. 6, an exemplary population selection interface is illustrated. In this example, the population parameter data on which the care management system 102 filters the member data 214 includes demographic data (e.g., age), general medical conditions, insurance claims, clinical history, SDoH risk scores, and SDoH needs. The population parameter data also includes a population ID, name, severity, visibility level, and status in this example, although other types and/or number of filters and/or population parameter data can also be used in other examples. Thus, selection and editing of the population parameter data allows a care manager to customize and personalize member populations for specified programs.

Referring back to FIG. 3, in step 306, the care management system 102 orchestrates opt-in, assessment, active journey, and exit journey phases to generate a digital journey (i.e., a multistep automated digital campaign for member outreach) for the program created in step 302 and populations identified in step 304. The care management system 102 provides a subset of the GUIs 210 to one of the care manager devices 110(1)-110(a), which are used to obtain orchestration data that defines automated engagement with the members of the identified population. In some examples, the defined automated engagement corresponds to delivery time for check-in messages, assessments, and/or digital educational content, as stored in the orchestration data.

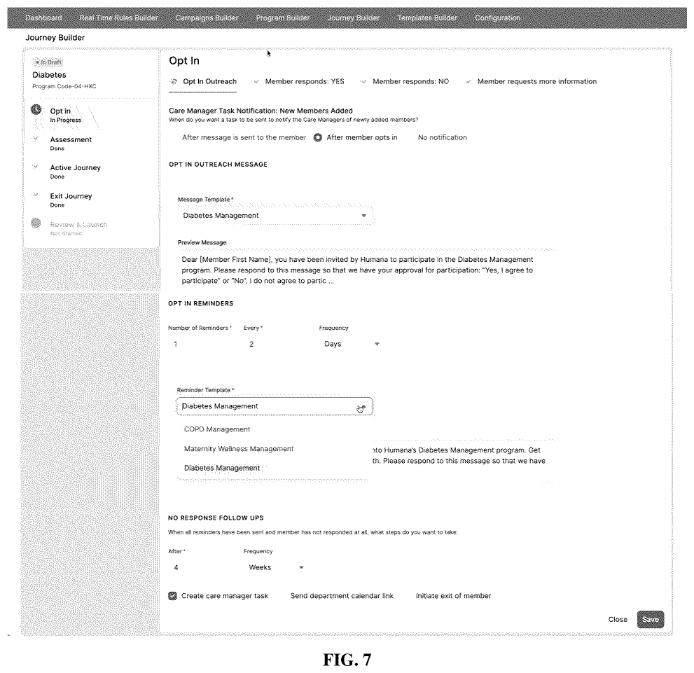

Referring now to FIG. 7, an exemplary opt-in orchestration interface for use in obtaining the orchestration data for the opt-in phase of the journey is illustrated. In this example, the orchestration established by the care manager using one of the care manager devices 110(1)-110(a) to interact with the opt-in orchestration interface includes opt-in outreach, positive response, negative response, and information request, although other conditions can also be used. The conditions to be orchestrated are available via tabs in the opt-in orchestration interface, one or more of which includes a message input field populated in response to selection by one of the care manager devices 110(1)-110(a) of one of the templates 212 (e.g., an enterprise-specific template library) and including stored text associated with the selected one of the templates 212.

For example, the care manager can select the “Diabetes Management” template to view a preview of a message to be sent during opt-in outreach. The text for the message can be retained or modified by the care manager and subsequently included in the stored orchestration data upon selection of the save button by the care manager. In addition to the opt-in message text, the orchestration data can include opt-in reminder data (e.g., number, frequency, and reminder text and/or an action to take when all reminders have been sent without a response from a member of the population). Similar orchestration can be established by the care manager for a response to positive response (e.g., a welcome message) or negative response (e.g., automatically exist the member from the population associated with the journey) from a member of the population or a request from a member of the population for more information (e.g., automatically store a task for the care manager to follow-up).

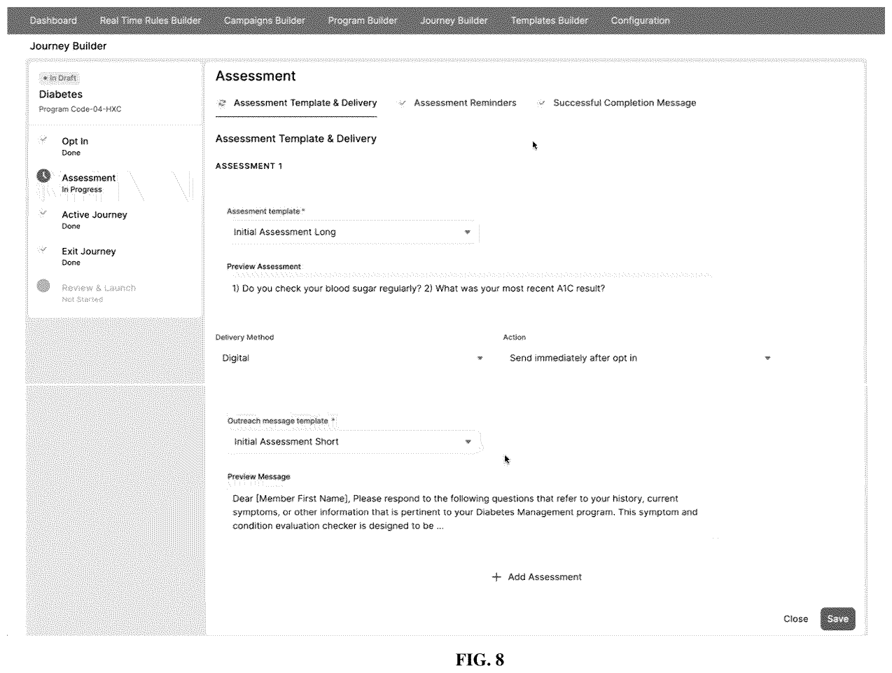

Referring to FIG. 8, an exemplary assessment delivery interface is illustrated. In this example, a care manager can user one of the care manager devices 110(1)-110(a) to select from a library of assessment templates (e.g., within templates 212), which can be previewed and modified. The assessment can be relatively short surveys that yield useful information for a care manager with respect to responding members of the population. The orchestration data for the assessment phase can also include a delivery method (e.g., short message service (SMS) message or application message) for the assessment, timing for the assessment, and the outreach message associated with the assessment. Additionally, orchestration can be facilitated for any number of assessments for the journey.

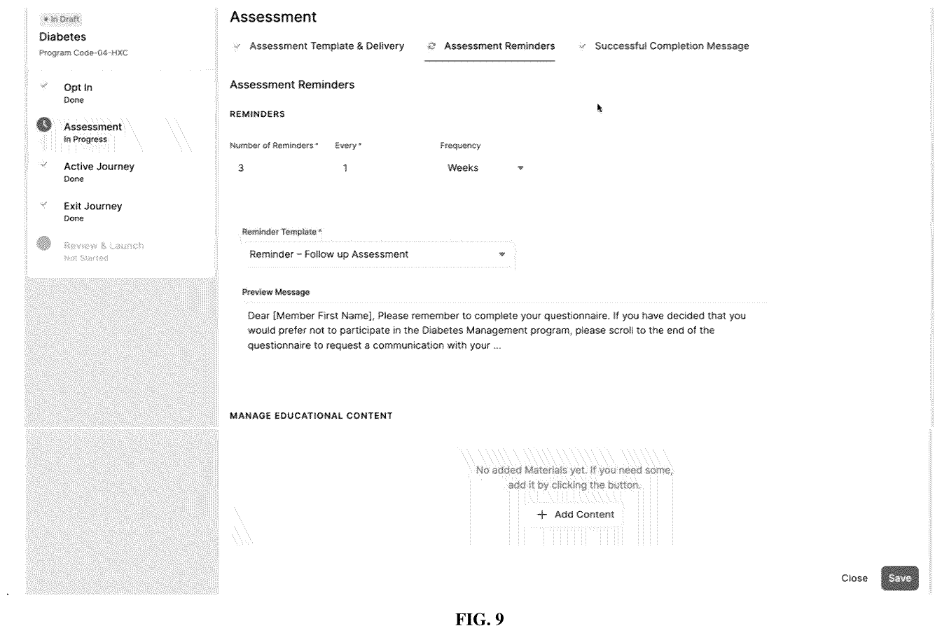

Referring to FIG. 9, an exemplary assessment reminder interface is illustrated. The assessment reminder interface can be used by a care manager to orchestrate assessment reminders by establishing the frequency and text (e.g., from templates 212) of the reminder messages. Optionally, the care manager can also upload digital educational content, which can be stored in a library in the memory 202 or obtained from third party source(s), for example. The education content can be sent along with the reminder message(s).

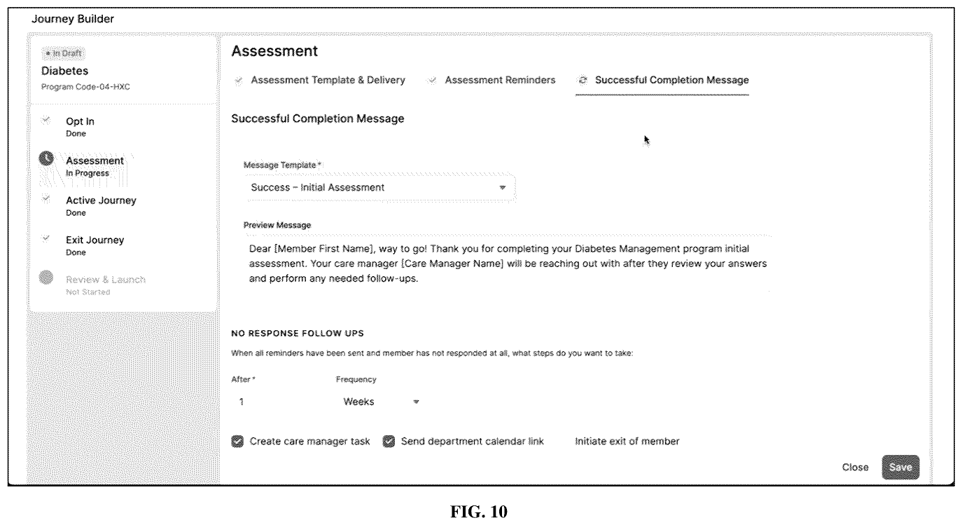

Referring to FIG. 10, an exemplary assessment completion interface is illustrated. The assessment completion interface has input fields for selection of a message template and customization of the association completion message that is sent automatically by the care management system 102 in response to receipt of a completed survey from one of the member devices 112(1)-112(b). The care manager again can orchestrate the automated response follow-ups after all reminder has been sent without assessment completion and can set establish in the orchestration data and automated generation of a care manager task, department calendar link, or exit of a member from the population associated with the journey, for example.

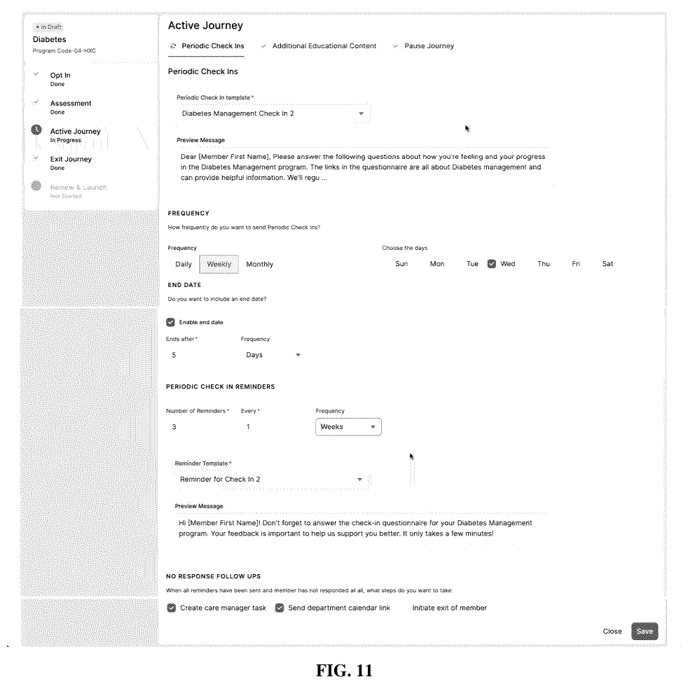

Referring to FIG. 11, an exemplary active journey check-in interface is illustrated. A care manager using one of the care manager devices 110(1)-110(a) can interact with the active journey check-in interface to orchestrate periodic check-ins as part of the journey outreach and automated engagement. More specifically, the care manager can establish the text of periodic check-in message via the templates 212, which can include message text for any number of disease states or conditions. The care manager can also establish the delivery method including frequency of the check-in message(s) and the activity to occur after all reminders have been sent without response from a member of the population.

Thus, the orchestration data defines delivery, reminder message(s), and successful completion message(s) associated with the check-in messages in the active journey phase or the assessments in the assessment phase. The inputs via the active journey check-in interface can be stored in the orchestration data for subsequent use by the care management system 102 to automatically carry out the journey during execution.

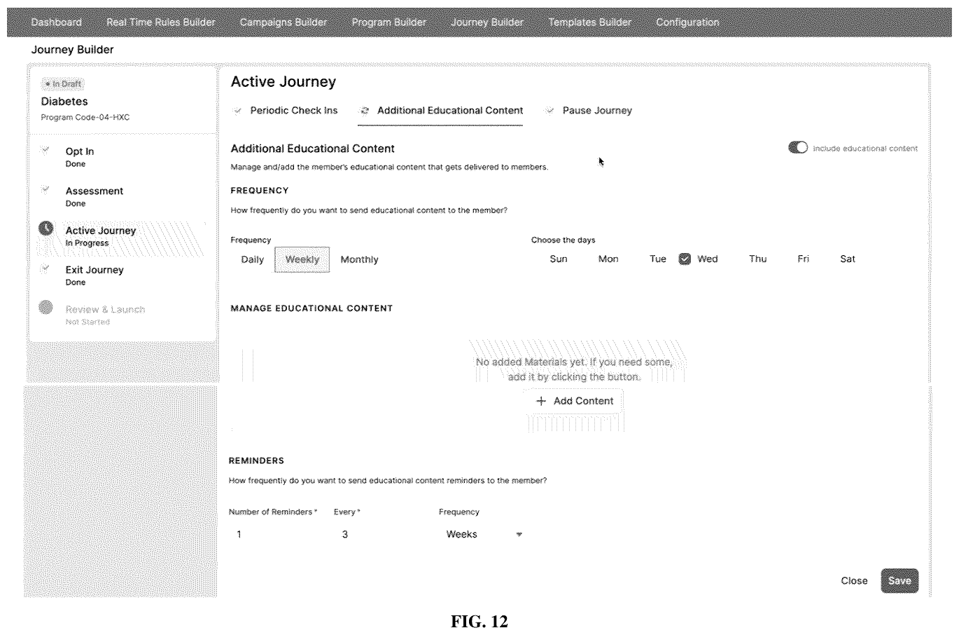

Referring now to FIG. 12, an exemplary active journey education content delivery interface is illustrated. The active journey education content delivery interface allows a care manager to orchestrate the delivery of digital educational content to the members of the population, including with respect to the frequency of the educational content, the educational content itself, and frequency of reminders regarding the educational content.

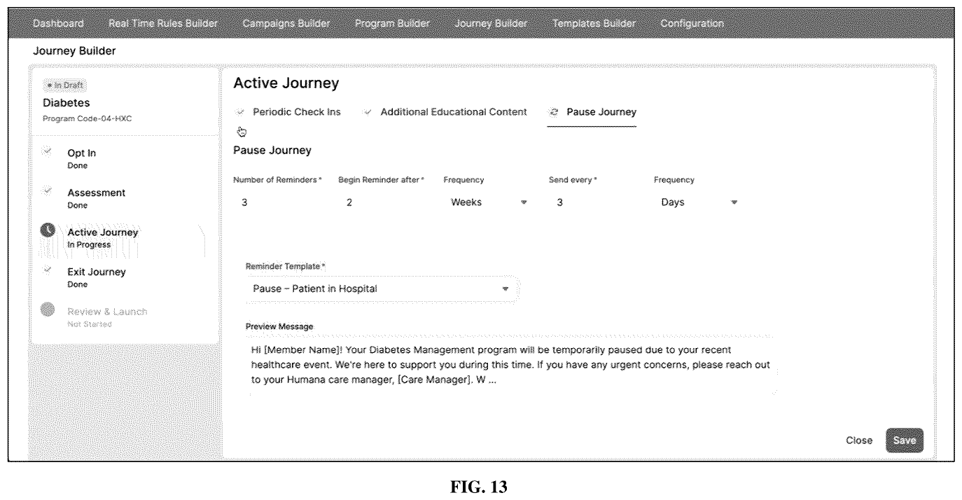

Referring to FIG. 13, an exemplary active journey pause interface is illustrated. With the active journey pause interface, a care manager can orchestrate a response to a request to pause the journey from one of the members of the population from one of the member devices 112(1)-112(b). The care manager can define reminder data regarding the paused journey, including the number, timing, and text content of the reminders, for example, which is stored in the orchestration data by the care management system 102.

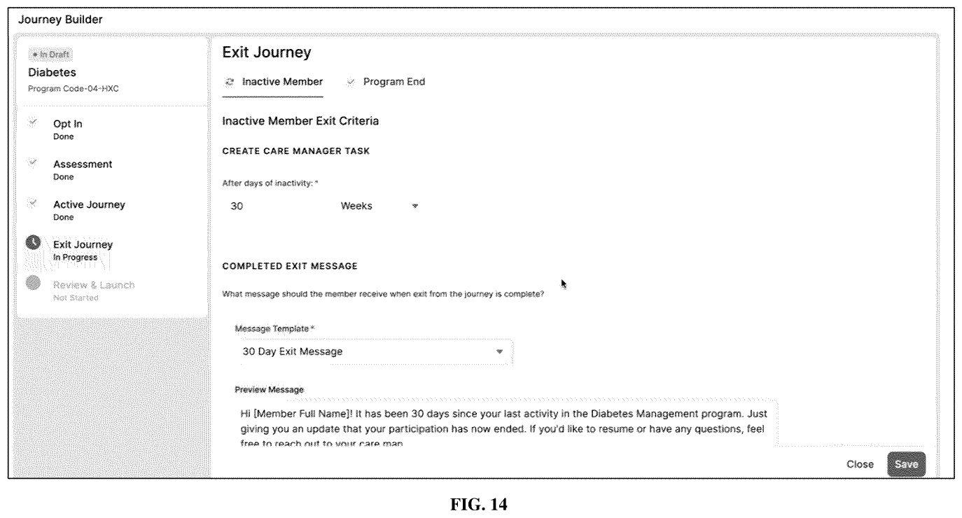

Referring to FIG. 14, an exemplary exit journey interface is illustrated. A care manager can interact with the exit journey interface in this example to establish the orchestration of an automated exit of a member of the population from the journey, including the time period of inactivity after which a member is automatically exited from the journey (e.g., the orchestration data is updated to remove an identifier for the member) and text of an exit message in response to a completion of the journey by a member. Additionally, the care manager can establish a fixed program end date and define text for an automated end message for the program, which is also stored in the orchestration data.

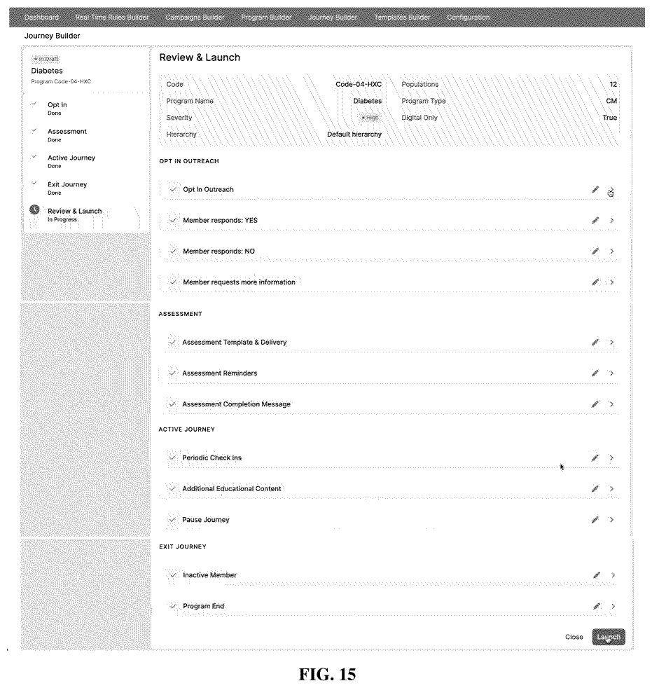

Referring to FIG. 15, an exemplary review and launch interface is illustrated. Optionally, the care management system 102 sends the review and launch interface to one of the care manager devices 110(1)-110(a), which includes a portion of the orchestration data and facilitates editing by the care manager of the orchestration data for any number of the journey phases to further customize the journey prior to execution of the journey.

Referring back to FIG. 3, in step 308, the care management system 102 executes the journey based on the orchestration data stored in step 306, which can include population data identifying the members to be included in the population for the journey. Accordingly, executing the journey in some examples includes sending via the communication network(s) 108 check-in messages, assessments, and/or educational content at selected times defined in the orchestration data to one or more of the member devices 112(1)-112(b) associated with one or more of the members identified in the orchestration data for the program.

In step 310, the care management system 102 determines whether an action has occurred with respect to one of the members of the population associated with the journey executed in step 308. In one example, the action can be submission of assessment data in response to automated provisioning of an assessment to one of the member devices 112(1)-112(b). In another example, the action can be automated receipt of third party data (e.g., ADT data) for the one of the members obtained via the communication network(s) 108 from one or more of the data provider servers 114(1)-114(c) as part of a periodic polling or request, for example. Other types of actions can also be identified in step 310. If the care management system 102 determines that an action has occurred with respect to one of the members, then the Yes branch is taken to step 312.

In step 312, the care management system 102 modifies the journey for the one of the members associated with the action based on the application of rule(s) stored in the memory 202 (e.g., as part of the rule engine 218). In some examples, the care management system 102 applies the stored rule(s) to obtained action data associated with the action determined in step 310 to generate additional orchestration data defining another automated engagement with the one of the members. The additional orchestration data can be a modification of the previously stored orchestration data associated with the journey.

For example, the care management system 102 may determine from obtained ADT data that one of the members was discharged from a hospital and can then automatically, based on the stored rule(s), modify the orchestration data for the member to send educational content relating to patient discharge in a particular number of days. Accordingly, the care management system 102 can dynamically adjust the journey for subsets of the population of members based on real-time activity, associated action data, and stored rule(s). After modifying the journey, or if the care management system 102 determines that an action has not occurred and the No branch is taken, then the care management system 102 proceeds to step 314.

In step 314, the care management system 102 determines whether a dashboard request has been received, such as from one of the care manager devices 110(1)-110(a) or one of the member devices 112(1)-112(b), for example. The dashboard request can be initiated via a web browser executing on one of those devices or a standalone application (e.g., a mobile application), for example, and other methods of initiating the dashboard request can also be used. If the care management system 102 determines that a dashboard request has been received, then the Yes branch is taken to step 316.

In step 316, the care management system 102 generates and outputs a care manager or member dashboard, which can be one of the GUIs 210. The care manager dashboard can allow a care manager to review recent activity performed, upcoming appointments, chat sessions, and/or prioritized worklists for members associate with the care manager. Additionally, the care manager dashboard can allow a care manager to view past and current programs, health summary details, demographic information, activity details, journey details, assessment submissions, and/or plan of care goal progress, for example, for individual members.

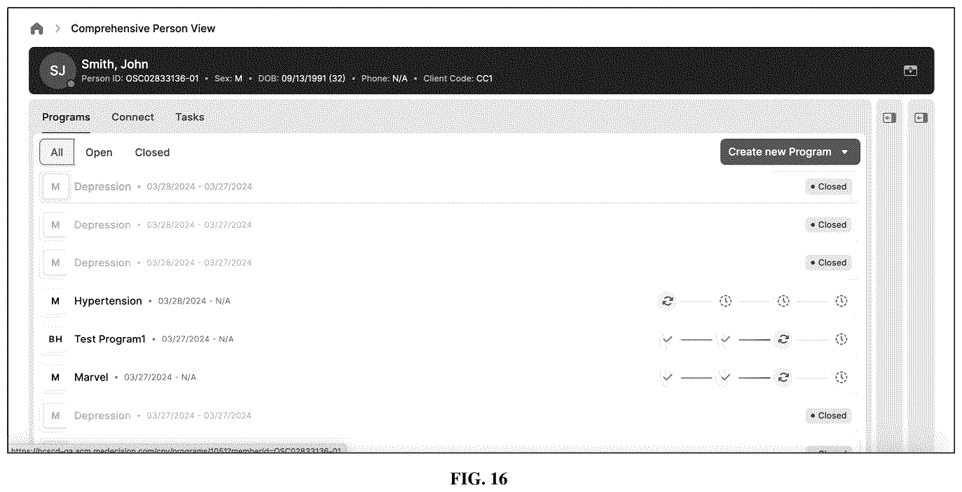

Referring to FIG. 16, an exemplary member program care manager dashboard is illustrated. In this example, a care manager can view historical and current program participation data for an individual member along with a high-level status with respect to the active programs. In some examples, the member program care manager dashboard includes expandable tab(s) that allow the care manager to view member data 214 (e.g., demographic data and/or health summary details) simultaneously with the current program participation data.

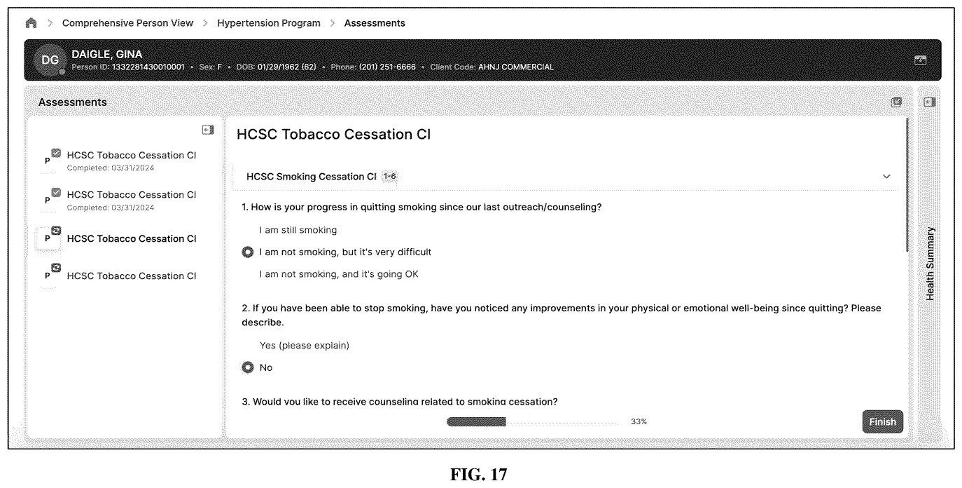

Referring to FIG. 17, an exemplary member assessment care manager dashboard is illustrated. The member assessment care manager dashboard allows a care manager to select and view open assessments and completed assessment responses for an individual member. Optionally, the member assessment care manager dashboard also can include expandable tabs as in the member program care manager dashboard. Other types of care manager dashboards including additional information such as journey details and/or member tasks, appointments, goals, chat sessions, and/or notifications, for example, are provided to the care manager devices 110(1)-110(a), can also be used in other examples.

The member dashboard in some examples allows a member to view upcoming tasks, complete assessments, view periodic check-in messages, engage in chat sessions with an associated care manager, monitor health goals, view educational content, and/or view or request appointments with an associated care manager, for example. Thus, the meaningful digital experiences orchestrated by the care manager as described above can be delivered through an application executed by the member devices 112(1)-112(b), for example, to inspire behavior change and maintain engagement between care managers and members. Comprehensive journeys and associated assessment advantageously enable early interventions for members and help members stay healthy.

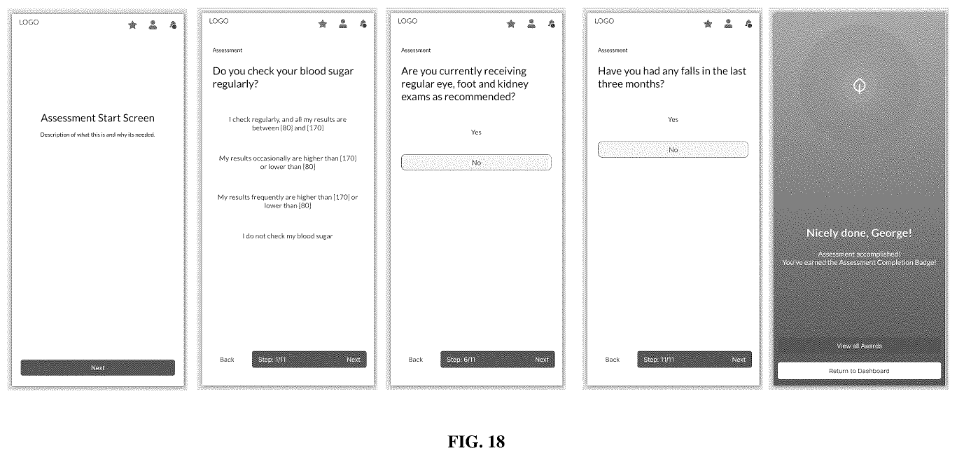

Referring to FIG. 18, an exemplary assessment member dashboard is illustrated. In this example, the assessment member dashboard is configured to update from an assessment start screen to assessment response submissions screens and finally an assessment completion screen. The assessment can be a template, or modified version thereof, orchestrated as described and illustrated above and with the responses made available by the care management system 102 to an associated care manager via the member assessment care manager dashboard.

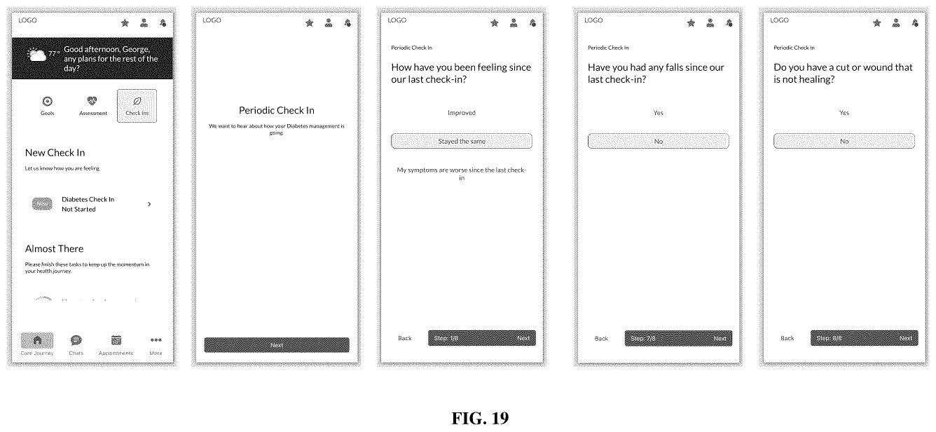

Referring to FIG. 19, an exemplary check-in member dashboard is illustrated. The check-in member dashboard includes an orchestrated check-in message as part of a journey. Member responses to check-in message can also be made available upon request to an associated care manager via a dashboard provided by the care management system 102 to one of the care manager devices 110(1)-110(a). Other types of member dashboards including additional information such as tasks, appointments, goals, chat sessions, and/or notifications, for example, and provided to the member devices 112(1)-112(b), can also be used in other examples.

Accordingly, the care management system 102 and GUIs 210 of this technology more effectively and efficiently facilitate member (e.g., patient) outreach. This technology efficiently orchestrates the delivery of omnichannel communications, assessments, and educational content generated using templates to facilitate customization of care management journeys to identified member populations. The automated digital care management journeys of this technology improve member engagement to facilitate better health outcomes for patients.

Having thus described the basic concept of the invention, it will be rather apparent to those skilled in the art that the foregoing detailed disclosure is intended to be presented by way of example only and is not limiting. Various alterations, improvements, and modifications will occur and are intended, though not expressly stated herein. These alterations, improvements, and modifications are intended to be suggested hereby, and are within the spirit and scope of the invention. Additionally, the recited order of processing elements or sequences, or the use of numbers, letters, or other designations, therefore, is not intended to limit the claimed processes to any order except as may be specified in the claims. Accordingly, the invention is limited only by the following claims and equivalents thereto.

What is claimed is:

1. A method for automated health care journey management, the method implemented by a care management system and comprising:
filtering stored member data based on obtained population parameter data to identify members to be associated with a program corresponding to a disease state or medical condition;
generating a journey for the program based on first orchestration data obtained from a care manager device via one or more graphical user interfaces (GUIs) provided to the care manager device via one or more communication networks, wherein the first orchestration data defines an orchestration of a first automated digital engagement with the members;
executing the journey based on the first orchestration data to, at least in part, initiate at least a portion of the first automated digital engagement;
determining that an action associated with one of the members has occurred;
modifying the journey for the one of the members by applying one or more stored rules to obtained action data associated with the action to generate second orchestration data defining an orchestration of a second automated digital engagement with the one of the members; and
executing the modified journey based on the second orchestration data to, at least in part, initiate at least a portion of the second automated digital engagement, wherein one or more of executing the journey for one or more of the members or executing the modified journey for the one of the members comprises sending via the communication networks one or more check-in messages, one or more assessments, or educational content at selected times to a member device associated with each of the one or more of the members or the one of the members, respectively.

2. The method of claim 1, further comprising:
obtaining base member data for the members, wherein the base member data comprises one or more of demographic, clinical, admission, discharge, and transfer (ADT) data, or social determinants of health (SDoH) needs data; and
generating enriched member data for another one or more of the members by at least analyzing the base member data to generate an SDoH risk score, wherein the stored member data comprises the base member data and the enriched member data.

3. The method of claim 1, wherein the first orchestration data is for opt-in, assessment, active journey, and exit journey phases of the journey and comprises a delivery time for each of the check-in messages, each of the assessments, or the educational content.

4. The method of claim 1, wherein one of the GUIs comprises a message input field populated in response to a selection received from the care manager device via the communication networks of a template and comprising stored text associated with the template.

5. The method of claim 4, wherein the one of the GUIs is configured to facilitate modification of the stored text to generate modified text and the first orchestration data comprises the modified text for at least one of the check-in messages or at least one of the assessments.

6. The method of claim 1, further comprising determining that the action has occurred in response to assessment data obtained from one of the member devices associated with the one of the members in response to one of the assessments or admission, discharge, and transfer (ADT) data for the one of the members obtained via the communication networks from one or more data provider servers.

7. The method of claim 1, wherein the first orchestration data defines delivery, one or more reminder messages, and one or more successful completion messages associated with at least one of the check-in messages or at least one of the assessments.

8. A care coordination system, comprising a memory having instructions stored thereon and one or more processors coupled to the memory and configured to execute the stored instructions to:
filter stored member data based on obtained population parameter data to identify members to be associated with a program corresponding to a disease state or medical condition;
generate a journey for the program based on first orchestration data obtained from a care manager device via one or more graphical user interfaces (GUIs) provided to the care manager device via one or more communication networks, wherein the first orchestration data defines an orchestration of a first automated digital engagement with the members;
execute the journey based on the first orchestration data to, at least in part, initiate at least a portion of the first automated digital engagement;
obtain third party data for one of the members via the communication networks from one or more data provider servers and via an application programming interface (API) provided by the one or more data provider servers;
determine that an action associated with one of the members has occurred based on the third party data;
modify the journey for the one of the members by applying one or more stored rules to obtained action data associated with the action to generate second orchestration data defining an orchestration of a second automated digital engagement with the one of the members; and
execute the modified journey based on the second orchestration data to, at least in part, initiate at least a portion of the second automated digital engagement, wherein one or more of executing the journey for one or more of the members or executing the modified journey for the one of the members comprises sending via the communication networks one or more check-in messages, one or more assessments, or educational content at selected times to a member device associated with each of the one or more of the members or the one of the members, respectively.

9. The care coordination system of claim 8, wherein the processors are further configured to execute the stored instructions to:
obtaining base member data for the members, wherein the base member data comprises one or more of demographic, clinical, admission, discharge, and transfer (ADT) data, or social determinants of health (SDoH) needs data; and
generating enriched member data for another one or more of the members by at least analyzing the base member data to generate an SDoH risk score, wherein the stored member data comprises the base member data and the enriched member data.

10. The care coordination system of claim 8, wherein the first orchestration data is for opt-in, assessment, active journey, and exit journey phases of the journey and comprises a delivery time for each of the check-in messages, each of the assessments, or the educational content.

11. The care coordination system of claim 8, wherein one of the GUIs comprises a message input field populated in response to a selection received from the care manager device via the communication networks of a template and comprising stored text associated with the template.

12. The care coordination system of claim 11, wherein the one of the GUIs is configured to facilitate modification of the stored text to generate modified text and the first orchestration data comprises the modified text for at least one of the check-in messages or at least one of the assessments.

13. The care coordination system of claim 8, wherein the third party data comprises admission, discharge, and transfer (ADT) data.

14. The care coordination system of claim 8, wherein the first orchestration data defines delivery, one or more reminder messages, and one or more successful completion messages associated with at least one of the check-in messages or at least one of the assessments.

15. A non-transitory computer readable medium having stored thereon instructions comprising executable code that, when executed by one or more processors, causes the processors to:
filter stored member data based on obtained population parameter data to identify members to be associated with a program corresponding to a disease state or medical condition;
generate a journey for the program based on first orchestration data obtained from a care manager device via one or more graphical user interfaces (GUIs) provided to the care manager device via one or more communication networks, wherein the first orchestration data defines an orchestration of a first automated digital engagement with the members;
execute the journey based on the first orchestration data to, at least in part, initiate at least a portion of the first automated digital engagement;
obtain admission, discharge, and transfer (ADT) data for one of the members via the communication networks from one or more data provider servers and via an application programming interface (API) provided by the one or more data provider servers;
determine that an action associated with one of the members has occurred based on the ADT data;
modify the journey for the one of the members by applying one or more stored rules to obtained action data associated with the action to generate second orchestration data defining an orchestration of a second automated digital engagement with the one of the members; and
sending via the communication networks one or more check-in messages, one or more assessments, or educational content at selected times to a member device associated with the one of the members to execute the modified journey based on the second orchestration data.

16. The non-transitory computer readable medium of claim 15, wherein the processors are further configured to execute the stored instructions to:
obtaining base member data for the members, wherein the base member data comprises one or more of demographic, clinical, admission, discharge, and transfer (ADT) data, or social determinants of health (SDoH) needs data; and
generating enriched member data for another one or more of the members by at least analyzing the base member data to generate an SDoH risk score, wherein the stored member data comprises the base member data and the enriched member data.

17. The non-transitory computer readable medium of claim 15, wherein the first orchestration data is for opt-in, assessment, active journey, and exit journey phases of the journey and comprises a delivery time for each of the check-in messages, each of the assessments, or the educational content.

18. The non-transitory computer readable medium of claim 15, wherein one of the GUIs comprises a message input field populated in response to a selection received from the care manager device via the communication networks of a template and comprising stored text associated with the template.

19. The non-transitory computer readable medium of claim 18, wherein the one of the GUIs is configured to facilitate modification of the stored text to generate modified text and the first orchestration data comprises the modified text for at least one of the check-in messages or at least one of the assessments.

20. The non-transitory computer readable medium of claim 15, wherein the first orchestration data defines delivery, one or more reminder messages, and one or more successful completion messages associated with at least one of the check-in messages or at least one of the assessments.
